SEQUENCE LISTING (3 sequences)

SEQ ID NO: 1 - protein, 68 aa, Artificial Sequence
  synthetic
KPVSLSYRCPCRFFESHVARANVKHLKILNTPNCACQIVARLKNNNRQVCIDPKLKWIQEYLEKCLNK

SEQ ID NO: 2 - protein, 38 aa, Artificial Sequence
  synthetic
MEGISIYTSDNYTEEMGSGDYDSMKEPAFREENANFNK

SEQ ID NO: 3 - protein, 68 aa, Homo sapiens
KPVSLSYRCPCRFFESHVARANVKHLKILNTPNCALQIVARLKNNNRQVCIDPKLKWIQEYLEKALNK

(19) United States
(12) Patent Application Publication
(10) Pub. No.: US 2021/0162009 A1
(43) Pub. Date: Jun. 3, 2021

(54) CHEMOKINE VARIANTS AS IMMUNE STIMULANTS

(71) Applicant: THE MEDICAL COLLEGE OF WISCONSIN, INC., Milwaukee, WI (US)

(72) Inventors: Michael DWINELL, Milwaukee, WI (US); Bryon JOHNSON, Milwaukee, WI (US); Brian VOLKMAN, Muskego, WI (US); Francis PETERSON, Racine, WI (US)

(73) Assignee: THE MEDICAL COLLEGE OF WISCONSIN, INC., Milwaukee, WI (US)

(21) Appl. No.: 16/770,544

(22) PCT Filed: Dec. 7, 2018
(86) PCT No.: PCT/US18/64493

§ 371 Date: Jun. 5, 2020

Related U.S. Application Data
(60) Provisional application No. 62/596,422, filed on Dec. 8, 2017.

(51) Int. Cl. A61K 38/19; A61K 45/06; A61P 35/00

(52) U.S. Cl. CPC A61K 38/195; A61P 35/00; A61K 45/06

(57) ABSTRACT

The present disclosure provides methods of using the CXCL12-α2 locked dimer polypeptide to mobilize cancer cells and hematopoietic stem cells into the bloodstream of a subject.

### CROSS-REFERENCE TO RELATED APPLICATIONS

This application claims priority to U.S. Provisional Application No. 62/596,422, filed Dec. 8, 2017, which is incorporated herein by reference in its entirety.

### STATEMENT REGARDING FEDERALLY SPONSORED RESEARCH

This invention was made with government support under grant numbers UO1-CA178960 and R01-AI058072 awarded by the National Institutes of Health. The government has certain rights in the invention.

### BACKGROUND

Chemokines are a superfamily of chemoattractant cytokine proteins which primarily serve to regulate a variety of biological responses and promote the recruitment and migration of multiple lineages of leukocytes and lymphocytes to a body organ tissue. Chemokines are classified into four families according to the relative position of the first two cysteine residues in the protein. In one family, the first two cysteines are separated by one amino acid residue (the CXC chemokines) and in another family the first two cysteines are adjacent (the CC chemokines). In a third family, the first two cysteines are separated by three amino acids (CX₃C chemokines). In a fourth family there is only one cysteine in the amino terminus (C chemokines).

The molecular targets for chemokines are cell surface receptors. One such receptor is CXC chemokine receptor 4 (CXCR4), which is a seven transmembrane G-protein coupled receptor (GPCR). CXCR4 is widely expressed on cells of hematopoietic and non-hematopoietic origin, and is a major co-receptor, along with CD4⁺, for certain strains of human immunodeficiency virus 1 (HIV-1).

CXCL12, formerly known as stromal cell-derived factor-1 (SDF-1), is an alpha or CXC type 7.8 kDa CXC chemokine. CXCL12 is the only known natural ligand for CXCR4 and directs homeostatic immune cell trafficking and inflammatory responses. Chemokine activation of specific GPCR directs cell migration toward higher chemokine concentration. High affinity CXCL12 binding requires the extracellular CXCR4 amino terminus. CXCL12 comprises two closely related members: CXCL12-α and CXCL12-β, the native amino acid sequences of which are known, as are the genomic sequences encoding these proteins (U.S. Pat. No. 5,563,048 and U.S. Pat. No. 5,756,084, both of which are incorporated by reference herein for all purposes). Stromal support cells of the bone marrow as well as cells of the liver, lung, gut and brain are known to produce CXCL12.

Chemokine activation of specific GPCR directs cell migration toward higher chemokine concentration. Originally described as a growth factor for bone marrow developing B cells, CXCL12 was subsequently characterized as a chemoattractant for T cells and monocytes. Genetic ablation of CXCR4 or CXCL12 is lethal, resulting from defects in hematopoiesis, vascularization of the intestines, cerebellar formation and heart development. Similar embryonic defects in either of those chemokine receptor or chemokine gene deficient animals has revealed roles for CXCR4-CXCL12 signaling in cardiovascular, neuronal, and hematopoietic stem cell development. Previous studies have also established a role for CXCL12 and CXCR4 in gut vascularization, a key process in mucosal immunity and homeostasis. In vitro, CXCL12 stimulates chemotaxis of a wide range of cells including monocytes and bone marrow derived progenitor cells. Particularly notable is its ability to stimulate a high percentage of resting and activated T-lymphocytes. CXCL12 also directs homeostatic immune cell trafficking and inflammatory responses.

Consistent with the fact that CXCR4 is a major co-receptor for HIV, CXCL12 has also been shown to block HIV entry into CD4+ T cells. CXCR4 is a co-receptor for T-tropic (X4) strains of HIV, which target CD4⁺ T cells, and CXCL12 can inhibit HIV-1 infection by preventing gp120 binding to CXCR4 and the subsequent gp41 mediated fusion. CXCR4 co-receptor usage correlates with AIDS onset, even though CCRS is the primary co-receptor for most HIV infections.

Efforts have been made to identify CXCL12-derived peptides that interfere selectively with HIV entry, and not with CXCL12 signaling. A wide range of potential CXCR4 binding fragments of CXCL12 have been proposed for use in blocking HIV infection, indicating that the anti-HIV activity of CXCL12, or fragments of CXCL12, does not depend on antagonism of the CXCR4 receptor.

Over the last 15 years it has become well established that CXCL12 directs the metastatic homing of cancer cells of the breast, colon, lung, pancreas, stomach, and other solid and hematologic malignancies. The ability of solid tumors to metastasize to distant organs reflects, in part, the epigenetic silencing of CXCL12 in malignant cancer cells that express CXCR4. High-risk hematologic cancers such as leukemia are known to express CXCR4 and it is believed that metastatic homing of these cells is similarly influenced by CXCL12 produced in the bone marrow, liver, lymph nodes and spleen.

Accordingly, there is a current need for cost-effective pharmaceutical agents and treatment methods for treating various conditions including blood cell deficiencies, cancers and other described conditions and to mobilize stem cells by manipulating and controlling CXCL12 and CXCR4.

### SUMMARY OF THE INVENTION

In a first aspect, provided herein is a method of mobilizing hematopoietic stem cells into the bloodstream of a subject by administering to the subject a therapeutically effective amount of a composition comprising a CXCL12-α2 locked dimer. In some embodiments, the CXCL12-α2 locked dimer comprises two monomers locked together. In some embodiments, at least one of the monomers has the amino acid sequence of SEQ ID NO:1. In some embodiments, the monomers are locked together by a disulfide linkage at residues 36 and 65 of SEQ ID NO:1. In some embodiments, the monomers are identical. In some embodiments, the monomers are not identical.

In some embodiments, the method may additionally comprise the step of harvesting the hematopoietic cells from the subject by apheresis. In some embodiments, the subject is human.

In a second aspect, provided herein is a method of mobilizing cancer cells into the bloodstream of a subject comprising administering to the subject a therapeutically effective amount of a composition comprising a CXCL12-α2 locked dimer. In some embodiments, at least one of the monomers has the amino acid sequence of SEQ ID NO:1. In some embodiments, the monomers are locked together by a disulfide linkage at residues 36 and 65 of SEQ ID NO:1. In some embodiments, the monomers are identical. In some embodiments, the monomers are not identical. In some embodiments, the subject is human. In some embodiments, the cancer expresses CXCR4.

In a third aspect, provided herein is a method of treating blood cancer in a subject comprising administering to the subject a therapeutically effective amount of a composition comprising a CXCL12-α2 locked dimer polypeptide, and administering to the subject a therapeutically effective amount of a chemotherapeutic agent. In some embodiments, at least one of the monomers has the amino acid sequence of SEQ ID NO:1. In some embodiments, the chemotherapeutic agent is selected from the group consisting of fludarabine, idarubicin, cytarabine, etoposide, cladribine, mitoxantrone, and venetoclax. In some embodiments, the cancer is selected from the group consisting of leukemia, lymphoma, and myeloma. In some embodiments, the cancer expresses CXCR4.

### BRIEF DESCRIPTION OF DRAWINGS

FIGS. 1A-1D demonstrate that CXCL12-α₂ (L36C/A65C) (CXCL12-α₂ or CXCL12 locked dimer) is a covalently locked dimer comprising two CXCL12 L36C/A65C sequences (SEQ ID NO: 1), each sequence comprising one subunit of the dimer. The lines connecting the cysteines show where the intra-and intermolecular disulfide bonds are. A) CXCL12-α₂ locked dimer amino acid sequence with the conserved intramolecular disulfide bonds (black lines) and the engineered intermolecular disulfide bonds (red lines) illustrated. B) SDS-PAGE of CXCL12-αand CXCL12-α₂ treated with and without dithiothreitol (DTT). CXCL12-α and CXCL12-α₂ migrate near the monomeric molecular weight of 8 kDa when treated with DTT. In contrast, CXCL12-α₂ migrates as a dimer while CXCL12-α migrates as a monomer in the absence of DTT. C) Translational diffusion measurements of CXCL12-α₂ indicate CXCL12-α₂ is dimeric. Diffusion coefficients (Dₛ) of wild-type CXCL12 (black circles) in 20 mM sodium phosphate at pH 7.4 plotted versus chemokine concentration. Non-linear fitting of the CXCL12-α Dₛ values indicates a dimer dissociation K_{d} of 120 μM with a pure monomer Dₛ value of ˜1.6 (×10⁻⁶ cm²s⁻¹) and a dimer value of ˜1.0 (×10⁻⁶ cm²s⁻¹). Dₛ values for 10, 50, and 150 μM SDF1₂ (red triangles) range from 1.08-1.09 (×10⁻⁶ cm²s⁻¹) consistent with those expected for CXCL12-α in the dimeric state. D) N-terminal peptides corresponding to the first thirty-eight amino acids of CXCR4 are illustrated. The sequence for p38 is identical to that of CXCR4 except for an additional N-terminal Gly-Ser dipeptide (cloning artifact) and the C28A substitution (green) introduced to prevent oxidative peptide dimer formation. The sulfated peptides are identical to p38 except for the inclusion of sulfotyrosine at position 21 for p38-sY₁ and at positions 7, 12 and 21 for p38-sY₃.

FIG. 2 demonstrates 50 μM CXCL12 locked dimer mobilization of malignant multiple myeloma tumor cells. Representative flow cytometry plots are on the left measuring side scatter counts (SSC) and GFP+-5T33 multiple myeloma cells in the circulation before and after treatment shown in the right panels. n=5 mice per treatment group.

FIGS. 3A-3B demonstrate the chemokine paradox: loss of migration at high concentrations. A) Chemotaxis follows a narrow biphasic dose response (green-to-red line) while G protein signaling measured by intracellular Ca2+-flux is able to be saturated (dotted green line). B) Structures of CXCL12 locked monomer and locked dimer. Native and engineered disulfide bonds are shown in red and blue, respectively.

FIGS. 4A-4C show that the CXCL12 locked dimer functionally activates CXCR4. A) The affinity of CXCL12 variants for CXCR4 was determined by ¹²⁵I-CXCL12 displacement. Kd values for binding of wild-type CXCL12 (CXCL12WT), L12 monomer (CXCL12H25R), and the CXCL12 locked dimer (CXCL12₂) were calculated as 25, 25, and 150 nM, respectively. B) Radioligand displacement of CXCR7-bound ¹²⁵I-CXCL12 resulted in weaker affinity of L12 dimer (Kd>1 mM) compared to WT (Kd≥28 nM) and L12 monomer (Kd≥15 nM). C) Intracellular calcium mobilization of cells stimulated by 10 nM CXCL12 variants was measured in the presence or absence of the Gαi inhibitor, pertussis toxin (PTX). From Drury, PNAS, 2011.

FIGS. 5A-5B show the effect of the CXCL12 locked dimer on melanoma metastasis. A) CXCL12 locked dimer dose dependently inhibits melanoma metastasis. B) At equimolar concentrations CXCL12 locked dimer is more effective than plerixafor/AMD3100. Modified from Takekoshi, Mol. Canc. Ther., 2012.

FIGS. 6A-6C show mobilization of lymphocytes into the peripheral blood (A) and out of the bone marrow (B) 1 hour after subcutaneous injection of 5 μM (0.8 mg/kg) CXCL12 locked dimer 104 or 4000 μM (20 mg/kg) plerixafor/AMD3100 102. Control mice received an injection of the chemokine vehicle (Veh), sterile PBS 100. Values are mean±SEM, n=3-4 mice per group. Asterisks (****)=P≤0.001. FIG. 6C shows mobilization of CXCR4+CD34+ hematopoietic stem cells using the CXCL12 locked dimer.

FIG. 7 shows limited toxicity of the CXCL12 locked dimer. Serum levels of aspartate aminotransferase (AST) and alanine aminotransferase (ALT) were measured using serologic assays to measure hepatic toxicity. Serum levels of blood urea nitrogen (BUN) were monitored to detect kidney damage.

FIGS. 8A-8C show mobilization of hematopoietic stem cells by the CXCL12 locked dimer. Mice were bled (baseline) prior to subcutaneous injection of LD at a final concentration of 5 μM. Mice were re-bled 15-minutes (FIG. 8A), 2-hours (FIG. 8B) or 4-hours (FIG. 8C) later. Two separate cohorts of mice (shown by circles and squares) were treated (n=3 per group) and show consistent increases in the number of CD34+ stem cells in the circulation.

### DETAILED DESCRIPTION OF THE INVENTION

All publications, patents, and patent applications mentioned in this specification are herein incorporated by reference to the same extent as if each individual publication, patent, and patent application was specifically and individually indicated to be incorporated by reference.

The present disclosure describes methods of use for the CXCL12 locked dimer polypeptide.

Before the present materials and methods are described, it is understood that this invention is not limited to the particular methodology, protocols, materials, and reagents described, as these may vary. It is also to be understood that the terminology used herein is for the purpose of describing particular embodiments only and is not intended to limit the scope of the present invention.

It must be noted that as used herein and in the appended claims, the singular forms “a”, “an”, and “the” include plural references unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described. All publications and patents specifically mentioned herein are incorporated by reference for all purposes including describing and disclosing the materials, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

CXCL12-α2 Locked Dimer Polypeptide. In one embodiment, the invention provides methods of using a CXCL12-α2 locked dimer polypeptide comprising at least two monomers. The monomers may be identical or may be non-identical. In one embodiment, at least one of the monomers has the amino acid sequence according to SEQ ID NO:1. In alternate embodiments, both monomers have the amino acid sequence according to SEQ ID NO:1. The CXCL12α2 locked dimer polypeptide is also described in U.S. Pat. Nos. 9,346,871, 8,524,670, and 7,923,016, each of which is incorporated herein as if set forth in its entirety.

By “locked” we mean the monomer components of the polypeptide are linked to each other via at least one covalent bond. The monomer and dimer forms do not interconvert. In a preferred embodiment, at least one of residues L36 and A65 of the wild type CXCL12 monomer sequence

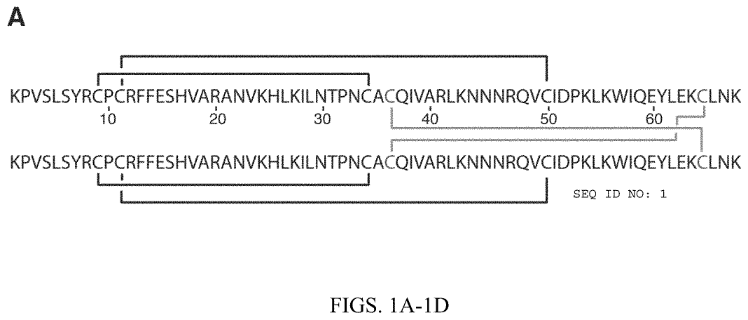

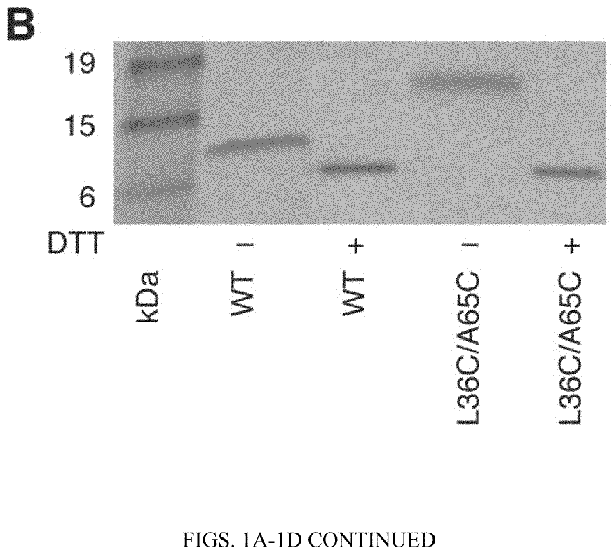

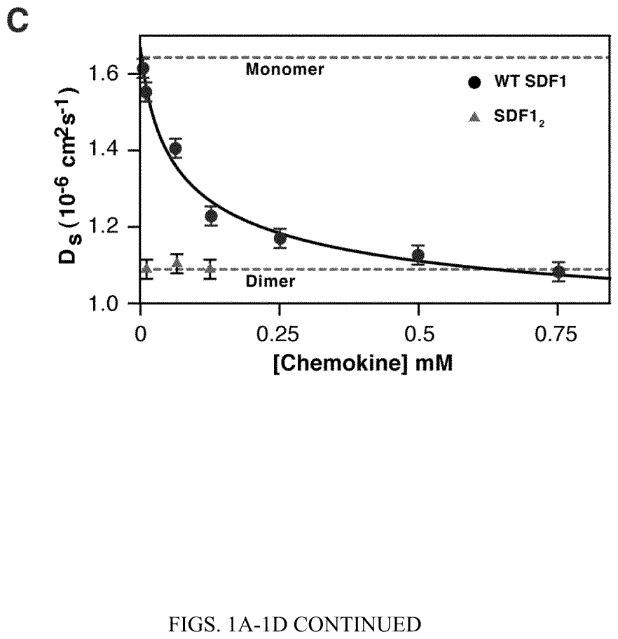

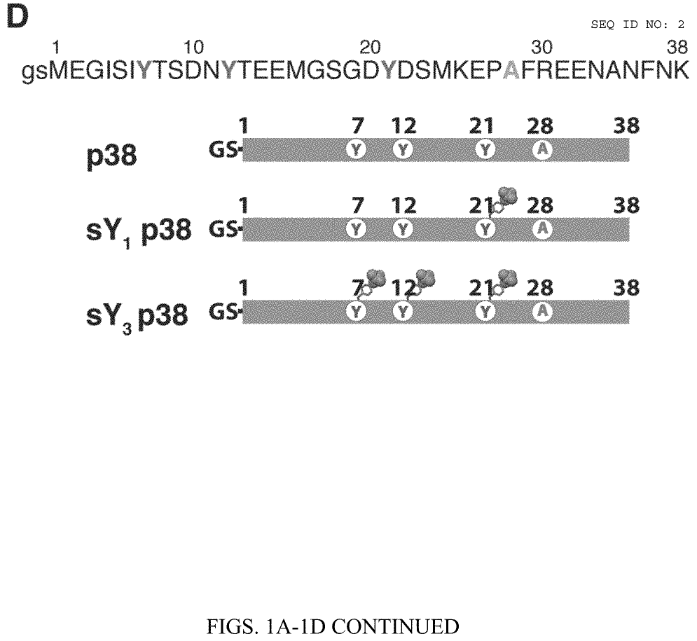

(KPVSLSYRCPCRFFESHVARANVKHLKILNTPNCALQIVARLKNNNRQVCIDPKLKWIQ EYLEKALNK, SEQ ID NO:3) is replaced with cysteine residues to create at least one intermolecular disulfide bond between cysteine residues at position 36 of one subunit and/or position 65 of the other monomer subunit. As shown in FIG. 1A, either or both cysteine residues at positions L36 and A65 can be replaced with cysteines to form the locked dimer with at least one, but preferably two, disulfide bonds.

Other residue(s) besides L36 and A65 in CXCL12 (SEQ ID NO:3) could be mutated to cysteines in order to form the locked dimer similar to the one of the present invention. For instance, a locked dimer can be created by mutating amino acid(s) in the CXCL12 dimer interface to cysteines that are positioned opposite one another yielding a disulfide bond that covalently links two CXCL12 monomers. For example, residue K27 is directly across the CXCL12 dimer interface from residue K27 of the opposing subunit and K27C mutation would likely make a locked dimer. Residues L26 and 128 are also on the CXCL12 dimer interface, and a L26C/I28C variant should form a locked dimer with L26C of one monomer subunit forming a disulfide bond with I28C of the opposing subunit and I28C of one monomer subunit forming a disulfide bond with L26C of the opposing subunit. All proposed cysteine mutations are numbered relative to SEQ ID NO:3.

In a preferred embodiment, the CXCL12-α2 locked dimer (SEQ ID NO:1) of the present invention has substitutions at both L36C/A65C residues relative to SEQ ID NO:3. Residue L36 is on beta strand 2 and A65 is near the end of the alpha helix of the dimer. Thus, disulfide bonds that form between beta strand 2 and the end of the helix generate the locked dimer. A similar locked dimer could be created using disulfide bonds introduced between beta strand 1 and the middle of the alpha helix. For example, CXCL12 with I28C/Y61C or I28C/L62C would form a locked dimer with beta strand one of one monomer having a disulfide bond to the middle of the alpha helix of the second monomer thus making a locked dimer. Additionally, a locked dimer may be created by generating a construct that produces two CXCL12 monomers where the C-terminus of one is linked to the N-terminus of the other through an amino acid linker.

Additional methods for making locked dimers of CXCL12 could also include other types of covalent linkages besides disulfide bonds including, but not limited to, chemical cross-linking reagents.

In a preferred embodiment, the locked dimer of the present invention comprises a substantially pure preparation. By “substantially pure” we mean a preparation in which more than 90%, e.g., 95%, 98% or 99% of the preparation is that of the locked dimer.

In a preferred embodiment, at least one of the monomers comprising the locked dimer of the present invention has the amino acid sequence as shown in SEQ ID NO:1 or a homologue or fragment thereof. In a further preferred embodiment, the dimer comprises two monomers having the amino acid sequence as shown in SEQ ID NO:1 or a homologue or variant thereof. By “homologue” we mean an amino acid sequence generally being at least 80%, preferably at least 90% and more preferably at least 95% identical to the polypeptide of SEQ ID NO:1 over a region of at least twenty contiguous amino acids. By “fragment,” we mean peptides, oligopeptides, polypeptides, proteins and enzymes that comprise a stretch of contiguous amino acid residues, and exhibit substantially a similar, but not necessarily identical, functional activity as the complete sequence. Fragments of SEQ ID NO:1, or their homologues, will generally be at least ten, preferably at least fifteen, amino acids in length, and are also encompassed by the term “a CXCL12 monomer” as used herein.

Mutations known to prevent degradation of CXCL12 or to increase the in vivo half-life may also be incorporated into the CXCL12-α2 sequence. For instance, adding a serine to the N-terminus along with a S4V substitution prevents CXCL12 degradation by proteases. Therefore, adding a serine to the N-terminus would likely similarly prevent protease degradation of the CXCL12-α2 locked dimer of the present invention.

Further, in addition to binding CXCR4, CXCL12 also binds to heparin found in the extracellular matrix on cell surfaces. The inventors have shown that the CXCL12-α2 locked dimer of the present invention also binds heparin. Amino acid substitutions in CXCL12, including K24S, K27S, or K24S/K27S can prevent heparin binding and increase the half-life of CXCL12 in vivo; therefore, similar mutations in CXCL12-α2 would likely prevent heparin binding and increase the in vivo half-life of the dimer.

CXCL12-α2 variants have been generated that have a Gly-Met dipeptide on the N-terminus. N-terminal extensions to CXCL12 prevent CXCR4 activation and their presence in CXCL12-α2 may increase its effectiveness. Additionally, it may be useful to create CXCL12-α2 variants where both subunits are not identical. For example, only one monomer of the CXCL12-a2 dimer may need to include an added N-terminal serine and a S4V substitution or the K24S, K27S, or K24S/K27S substitutions to prevent heparin binding. Alternatively, a CXCL12-α2 variant where the N-terminus of one monomer has the native sequence but the other has been extended may have different or enhanced pharmacological properties compared to CXCL12-α2.

The locked CXCL12 dimer could also be incorporated into a larger protein or attached to a fusion protein that may function to increase the half-life of the dimer in vivo or be used as a mechanism for time released and/or local delivery (U.S. Patent Appl. No. 20060088510).

In another embodiment, the invention provides an isolated CXCL12-α2 locked dimer polypeptide as described above. By “isolated” we mean a nucleic acid sequence that is identified and separated from at least one component or contaminant with which it is ordinarily associated. An isolated nucleic acid is present in a form or setting that is different from that in which it is found in nature. In contrast, non-isolated nucleic acids such as DNA and RNA are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, an isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide can be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide can be single-stranded), but can contain both the sense and anti-sense strands (i.e., the oligonucleotide or polynucleotide can be double-stranded).

CXCL12-α2 locked dimer polypeptides of the present invention can be prepared by standard techniques known in the art. The peptide component of CXCL12-α2 is composed, at least in part, of a peptide, which can be synthesized using standard techniques such as those described in Bodansky, M., Principles of Peptide Synthesis, Springer Verlag, Berlin (1993) and Grant, G. A. (ed.). Synthetic Peptides: A User's Guide, W. H. Freeman and Company, New York (1992). Automated peptide synthesizers are commercially available (e.g., Advanced ChemTech Model 396; Milligen/Biosearch 9600). Additionally, one or more modulating groups can be attached to the CXCL12-α2 derived peptidic component by standard methods, such as by using methods for reaction through an amino group (e.g., the alpha-amino group at the amino-terminus of a peptide), a carboxyl group (e.g., at the carboxy terminus of a peptide), a hydroxyl group (e.g., on a tyrosine, serine or threonine residue) or other suitable reactive group on an amino acid side chain (see e.g., Greene, T. W. and Wuts, P. G. M. Protective Groups in Organic Synthesis, John Wiley and Sons, Inc., New York (1991)). Exemplary syntheses of preferred CXCL12-α2 locked dimer polypeptides according to the present invention are described further in the Examples below.

Peptides of the invention may be chemically synthesized using standard techniques such as those described in Bodansky, M. Principles of Peptide Synthesis, Springer Verlag, Berlin (1993) and Grant, G. A. (ed.). Synthetic Peptides: A User's Guide, W. H. Freeman and Company, New York, (1992) (all of which are incorporated herein by reference).

In another aspect of the invention, peptides may be prepared according to standard recombinant DNA techniques using a nucleic acid molecule encoding the peptide. A nucleotide sequence encoding the peptide can be determined using the genetic code and an oligonucleotide molecule having this nucleotide sequence can be synthesized by standard DNA synthesis methods (e.g., using an automated DNA synthesizer). Alternatively, a DNA molecule encoding a peptide compound can be derived from the natural precursor protein gene or cDNA (e.g., using the polymerase chain reaction (PCR) and/or restriction enzyme digestion) according to standard molecular biology techniques.

CXCL12-α2 Locked Dimer Polypeptide Pharmaceutical Compositions. In another embodiment, the invention provides a method of using a composition comprising a substantially pure CXCL12-α2 locked dimer polypeptide of the present invention, and a pharmaceutically acceptable carrier. By “pharmaceutically acceptable carrier” we mean any and all solvents, dispersion media, coatings, antibacterial and antifungal agents, isotonic and absorption delaying agents, and the like that are physiologically compatible. In one embodiment, the carrier may be suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, membrane nanoparticle or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, such as, monostearate salts and gelatin.

Moreover, the CXCL12-α2 locked dimer polypeptide of the present invention can be administered in a time-release formulation, such as in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g. the CXCL12-α2 locked dimer) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. The CXCL12-α2 locked dimer polypeptide of the present invention also may be formulated with one or more additional compounds that enhance the solubility of the CXCL12-α2 locked dimer polypeptide.

Administration. The CXCL12-α2 locked dimer polypeptide of the present invention, optionally comprising other pharmaceutically active compounds, can be administered to a patient orally, rectally, parenterally, (e.g., intravenously, intramuscularly, or subcutaneously) intracisternally, intravaginally, intraperitoneally, intravesically, locally (for example, powders, ointments or drops), or as a buccal or nasal spray. Other contemplated formulations include projected nanoparticles, liposomal preparations, resealed erythrocytes containing the active ingredient, and immunologically-based formulations.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound, and the like. In particular, parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the CXCL12-α2 locked dimer of the invention combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols (e.g., propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the CXCL12-α2 locked dimer polypeptide is provided in dry (i.e., powder or granular) form for reconstitution with a suitable vehicle (e.g., sterile pyrogen-free water) prior to parenteral administration of the reconstituted composition.

The pharmaceutical compositions can be prepared, packaged, or sold in the form of a sterile injectable aqueous or oily suspension or solution. This suspension or solution can be formulated according to the known art. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation can comprise pharmaceutically acceptable polymeric or hydrophobic materials such as an emulsion, an ion exchange resin, a sparingly soluble polymer, or a sparingly soluble salt.

The CXCL12-α2 locked dimer polypeptide of the present invention may also contain adjuvants such as suspending, preserving, wetting, emulsifying, and/or dispersing agents, including, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, such as aluminum monostearate and/or gelatin.

Dosage forms can include solid or injectable implants or depots. In preferred embodiments, the implant comprises an effective amount of the α2 locked dimer polypeptide and a biodegradable polymer. In preferred embodiments, a suitable biodegradable polymer can be selected from the group consisting of a polyaspartate, polyglutamate, poly(L-lactide), a poly(D,L-lactide), a poly(lactide-co-glycolide), a poly(ϵ-caprolactone), a polyanhydride, a poly(beta-hydroxy butyrate), a poly(ortho ester) and a polyphosphazene. In other embodiments, the implant comprises an effective amount of CXCL12-α2 locked dimer polypeptide and a silastic polymer. The implant provides the release of an effective amount of CXCL12-α2 locked dimer polypeptide for an extended period ranging from about one week to several years.

Solid dosage forms for oral administration include capsules, tablets, powders, and granules. In such solid dosage forms, the CXCL12-α2 locked dimer polypeptide is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the active ingredient can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets can be made by molding, in a suitable device, a mixture of the active ingredient, a pharmaceutically acceptable carrier, and at least sufficient liquid to moisten the mixture.

Tablets may be manufactured with pharmaceutically acceptable excipients such as inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or coated using known methods to achieve delayed disintegration in the gastrointestinal tract of a human, thereby providing sustained release and absorption of the active ingredient. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols, and the like. Hard capsules comprising the active ingredient can be made using a physiologically degradable composition, such as gelatin. Such hard capsules comprise the active ingredient, and can further comprise additional ingredients including, for example, an inert solid diluent such as calcium carbonate, calcium phosphate, or kaolin. Soft gelatin capsules comprising the active ingredient can be made using a physiologically degradable composition, such as gelatin. Such soft capsules comprise the active ingredient, which can be mixed with water or an oil medium such as peanut oil, liquid paraffin, or olive oil.

Dose Requirements. In particular embodiments, a preferred range for therapeutically or prophylactically effective amounts of CXCL12-α2 locked dimer polypeptide may be 0.1 nM-1.0μM as administered. It is to be noted that dosage values may vary with the severity of the condition to be alleviated. It is to be further understood that for any particular patient, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

The amount of CXCL12-α2 locked dimer polypeptide in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such as active compound for the treatment of sensitivity in individuals.

Methods of Use. The invention also provides corresponding methods of use, including methods of medical treatment, in which a therapeutically effective dose of a CXCL12-α2 locked dimer polypeptide, preferably wherein the dimer comprises at least one monomer having the amino acid sequence according to SEQ ID NO:1, is administered in a pharmacologically acceptable formulation. Accordingly, the invention also provides therapeutic compositions comprising a CXCL12-α2 locked dimer polypeptide and a pharmacologically acceptable excipient or carrier, as described above. The therapeutic composition may advantageously be soluble in an aqueous solution at a physiologically acceptable pH.

In one embodiment, the invention provides a method of mobilizing malignant hematologic cells in a patient in need thereof as a treatment for blood cancer. By “blood cancer” we mean cancers that affect the production or function of hematopoietic lymphoid or myeloid-derived cells. As defined by the American Society of Hematology these cancers include leukemia, a type of cancer found in your blood and bone marrow, caused by the rapid production of abnormal white blood cells; lymphoma, a type of blood cancer that affects the lymphatic system and/or lymphocytes, a type of white blood cell that fight infection; and myeloma, a cancer of antibody-producing plasma cells. Such cancers include, but are not limited to, those that express CXCR4, including multiple myeloma, non-Hodgkin's lymphoma, acute or chronic myelogenous leukemia, and acute or chronic lymphoblastic leukemia. CXCR4-expressing blood cancers home to and take up residence and are protected from conventional clinical therapies in bone marrow, liver, spleen and other lymphatic tissues which produce its ligand. Treatment with CXCL12 a2 locked dimer will mobilize or otherwise block blood cancer homing to bone marrow, liver, spleen or other lymphatic tissues and be amenable to further treatment or to enhance engraftment with non-malignant donor white blood cells or hematopoietic stem cells administered as standard of care.

In some cases, the CXCL12-α2 locked dimer may be optionally administered in combination with one or more additional active agents. Such agents include chemotherapeutic agents. Any suitable combination of such active agents is also contemplated. When administered in combination with one or more active agents, the CXCL12 locked dimer can be administered either simultaneously or sequentially with other active agents. For example, a cancer subject may simultaneously receive the CXCL12 locked dimer and a chemotherapeutic agent for a length of time or according to a dosage regimen sufficient to support treatment, alleviation or remission of the cancer. In some embodiments, the CXCL12 locked dimer may be administered prior to the administration of a standard of care chemotherapeutic agent, including but not restricted to fludarabine, idarubicin, cytarabine, etoposide, cladribine, mitoxantrone, and venetoclax. In some embodiments, the CXCL12 locked dimer is administered concurrently with the chemotherapeutic agent.

In one embodiment, the invention provides a method of mobilizing hematopoietic stem cells in a subject. As used herein “hematopoietic stem and progenitor enriched cells” refers to CD34+ and CD34− hematopoietic cells capable of giving rise to both the myeloid and lymphoid lineages of blood cells. Mobilized hematopoietic cells and progenitor enriched cells in a subject will then be harvested by apheresis or another appropriate method known in the art. The harvested stem cells can be used for any suitable purpose such as, but not limited to, re-engraftment during autologous and/or allogeneic stem cell transplantation. In some embodiments, the hematopoietic stem cells to be mobilized express CXCR4. In some embodiments, the hematopoietic stem cells to be mobilized express CXCR4 and CD34. In some embodiments, the hematopoietic stem cells to be mobilized express at least one marker selected from the group consisting of CD34, CD11, SCA1, CD90, CD38, Lin and CXCR4.

In some embodiments, the CXCL12 locked dimer is administered as part of an autologous stem cell transplantation treatment. Following administration of CXCL12 to mobilize hematopoietic stem cells in a subject, the hematopoietic stem cells are harvested and processed, for example by peripheral blood apheresis. Following harvest or collection of the cells, the subject undergoes a myeloablative treatment. The harvested hematopoietic cells are then re-introduced into the patient wherein they engraft into the bone marrow of the subject. In some embodiments, at least about 5×10⁶ CD34+ cells/kg are harvested by apheresis prior to myeloablative treatment.

By “mobilization” we mean a detectable increase in the number of cells circulating in the bloodstream of the subject, e.g., at least about 100%, 150%, 200%, 250%, 300%, 400%, or 500% increase in the number of cells circulating following single or multiple injections of CXCL12. In some embodiments, administration of the CXCL12-α2 locked dimer may be administered in combination with G-CSF.

By “subject” we mean mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, fish and the like. The term “subject” does not denote a particular age or sex.

By “treating” we mean the management and care of a subject for the purpose of combating the disease, condition, or disorder. The terms embrace both palliative treatments and treatments to elicit a desired response in the subject. Treating includes the administration of a compound of the present invention to ameliorate and/or improve the onset of the symptoms or complications, alleviating the symptoms or complications, or eliminating the disease, condition, or disorder. Treating also includes the administration of a compound of the present invention to induce a desired phenotype or to invoke increase circulation of hematopoietic stem cells or cancer cells in the bloodstream.

By “ameliorate”, “amelioration”, “improvement” or the like we mean a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated with the locked dimer of the present invention, where the untreated subjects have, or are subject to developing, the same or similar disease, condition, symptom or the like. Amelioration of a disease, condition, symptom or assay parameter may be determined subjectively or objectively, e.g., self-assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of cell migration within a subject. Amelioration may be transient, prolonged or permanent or it may be variable at relevant times during or after the locked dimer of the present invention is administered to a subject or is used in an assay or other method described herein or a cited reference, e.g., within about 1 hour of the administration or use of the locked dimer of the present invention to about 3, 6, 9 months or more after a subject(s) has received the locked dimer of the present invention.

By “modulation” of, e.g., a symptom, level or biological activity of a molecule, replication of a pathogen, cellular response, cellular activity or the like means that the cell level or activity is detectably increased or decreased. Such increase or decrease may be observed in treated subjects as compared to subjects not treated with the locked dimer of the present invention, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more. Modulation may be determined subjectively or objectively, e.g., by the subject's self-assessment, by a clinician's assessment or by conducting an appropriate assay or measurement, including, e.g., quality of life assessments or suitable assays for the level or activity of molecules, cells or cell migration within a subject. Modulation may be transient, prolonged or permanent or it may be variable at relevant times during or after the locked dimer of the present invention is administered to a subject or is used in an assay or other method described herein or a cited reference, e.g., within about 1 hour of the administration or use of the locked dimer of the present invention to about 3, 6, 9 months or more after a subject(s) has received the locked dimer of the present invention.

By “administering” we mean any means for introducing the CXCL12-α2 locked dimer polypeptide of the present invention into the body, preferably into the systemic circulation. Examples include but are not limited to oral, buccal, sublingual, pulmonary, transdermal, transmucosal, as well as subcutaneous, intraperitoneal, intravenous, and intramuscular injection.

By “therapeutically effective amount” we mean an amount effective, at dosages and for periods of time necessary, to achieve the desired therapeutic result, such as mobilization of hematopoietic stem cells into the bloodstream or mobilization of blood cancer cells. A therapeutically effective amount of the CXCL12-α2 locked dimer polypeptide may vary according to factors such as the disease state, age, sex, and weight of the subject, and the ability of the CXCL12-α2 locked dimer polypeptide to elicit a desired response in the subject. Dosage regimens may be adjusted to provide the optimum therapeutic response. A therapeutically effective amount is also one in which any toxic or detrimental effects of the CXCL12-α2 locked dimer polypeptide are outweighed by the therapeutically beneficial effects.

The present invention has been described in terms of one or more preferred embodiments, and it should be appreciated that many equivalents, alternatives, variations, and modifications, aside from those expressly stated, are possible and within the scope of the invention.

### Example 1

The embodiment described in this example demonstrates that the CXCL12-α2 locked dimer mobilizes malignant multiple myeloma tumors cells and demonstrates mobilization relative to AMD3100 treatment.

The CXCR4 receptor has been linked with the metastasis of nearly 23 hematologic and solid cancers. Notably, multiple myeloma (MM) is an intractable hematological cancer with limited therapeutic options because these CXCR4-expressing tumors metastasize to the bone marrow where they are protected from conventional chemotherapy. The CXCL12 locked dimer in its activity as an immune stimulant will mobilize MM cells from the protective bone marrow niche or prevent metastatic return (relapse) to the bone marrow, making those cancer cells increasingly vulnerable to cytotoxic therapies. Data presented herein demonstrates the CXCL12 locked dimer mediated mobilization of malignant blood cells out of the protected environment of the bone marrow and into circulation where they are more sensitive to chemotherapy.

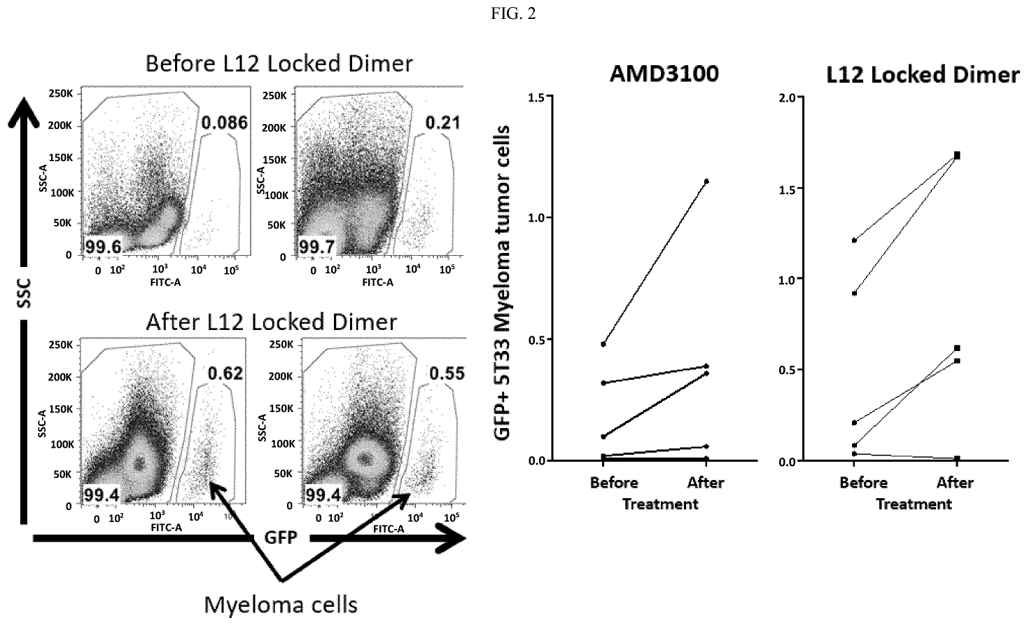

As shown in FIG. 2, administration of the CXCL12 locked dimer mobilizes multiple myeloma cells into the blood. This mobilization is compared to the mobilization following administration of AMD3100. Mice bearing GFP-tagged 5T33 multiple myeloma cells were bled immediately prior to subcutaneous injection of either the CXCL12 locked dimer (50 μM) or AMD3100 (4 mM). Individual mice were re-bled 1-hour later and GFP+ myeloma tumor cells in peripheral circulation quantified by flow cytometry. Representative flow cytometry plots are on the left with GFP+-5T33 multiple myeloma cells in the circulation before and after treatment shown in the right panels. n=5 mice per treatment group.

### Example 2

The embodiment described in this example demonstrates that the CXCL12 locked dimer mobilizes hematopoietic stem cells into the blood.

There are approximately 11,000 autologous stem cell transplants (ASCT) in the US annually to treat blood disorders like leukemia, multiple myeloma, and lymphoma. ASCT is widely performed as part of the treatment strategy in conjunction with chemotherapy and total body radiation for leukemia, multiple myeloma, and lymphoma. Chemotherapy and irradiation destroy cells in the bone marrow. The goal of ASCT is to replenish this organ with stem cells. Before the patient receives chemotherapy/radiation, blood is harvested from the patient and the stem cells in the blood are extracted (via apheresis) and stored. After chemotherapy/radiation, the stem cells are reinfused (engrafted) back into that patient where they colonize the bone marrow and restore the patient's normal blood cell production.

It is clear that cell graft survival depends on the number of stem cells engrafted. As many as 30% of ACST fail due to the inability to collect enough peripheral stem cells, raising the cost of treatment by nearly 80%. Current therapies like granulocyte colony stimulating factor (G-CSF) and the chemokine receptor antagonist plerixafor are aimed at increasing stem cell mobilization to the peripheral blood and have found limited success in enhancing cell numbers. However, these therapies still require multiple days of apheresis to collect enough HSCs for transplant and may stimulate splenomegaly or evoke secondary malignancies.

Successful ASCT depends on harvesting a sufficient number of peripheral stem cells to infuse back into the patient. The goal is to obtain 5×10⁶ CD34+ cells/kg weight of the donor/recipient. For this reason, patients are treated with drugs to “mobilize” stem cells from the bone marrow into the peripheral blood. Currently, there are 2 types of drugs in use: granulocyte colony stimulating factor (G-CSF) alone or in combination with the chemokine receptor antagonist AMD3100 (plerixafor). Broxmeyer et al. (Rapid mobilization of murine and human hematopoietic stem and progenitor cells with AMD3100, a CXCR4 antagonist, JEM, 2005, 201(8):1307-1318) suggest that these drugs may work synergistically.

Approved by the FDA in 2008 only for non-Hodgkin's lymphoma (NHL) and multiple myeloma (MM), plerixafor is believed to work by blocking the binding of CXCL12 to its CXCR4 receptor. This, in turn, alters traffic patterns and/or releases stem cells from the bone marrow into the circulating blood. Likewise, there is a strong interest in drugs that can mobilize tumor cells from their “niches” in the bone marrow, rendering them more susceptible to anti-cancer drugs. AMD3100 (plerixafor) and similar compounds are being studied as “chemosensitizing” agents.

The CXCL12 locked dimer binds strongly to the stem cell homing receptor CXCR4 and blocks the effect of CXCL12, its natural ligand. The CXCL12 locked dimer is an engineered variant of the natural chemokine. This recombinant protein is a partial agonist that binds CXCR4 and disrupts chemotactic cell migration. Data presented herein show that the CXCL12 locked dimer is substantially more potent than plerixafor, a first-generation CXCR4 antagonist. Administration of the CXCL12 locked dimer will increase the number of successful ASCTs and reduce the number of apheresis sessions necessary for cell collection. This will have secondary effects of reduced healthcare burden by decreasing hospital stays, reduce possible off-target effects common to current therapies, and, most importantly, increase the number of patients with successful HSC engraftment and functional hematological processes. Given the low physiologic concentration of CXCL12 locked dimer needed to see an effect, we expect fewer off-target effects, decreased end-organ damage, and limited adverse events.

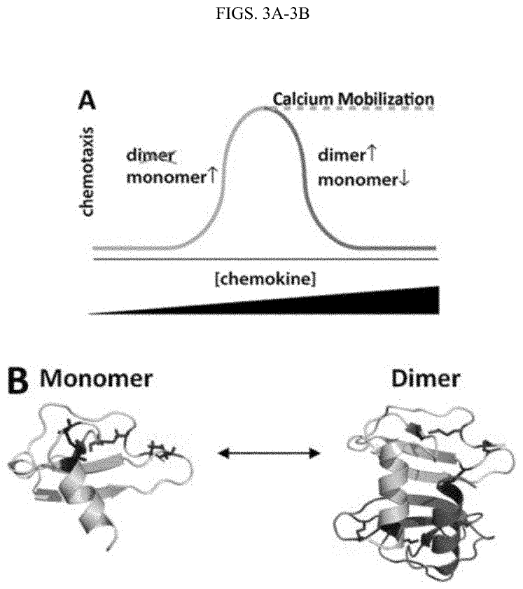

Engineered CXCL12 locked dimer stimulates a non-motile state—Basic research in chemokine structure and receptor signaling led to the discovery that the natural chemokine CXCL12 can either promote or arrest leukocyte migration, depending on the ligand concentration. The characteristic ‘bell-shaped’ chemotaxis profile diverges from the G-protein signaling response at high concentrations (FIG. 3A). Because most chemokines can exist as either protein monomers or dimers we speculated that concentration dependent changes in the oligomeric state of CXCL12 might be linked to dose-dependent migration. We used disulfide bond protein engineering to construct CXCL12 variants that are restricted to either the monomer or dimeric configuration (FIG. 3B). The naturally occurring CXCL12 dimer and its monomeric counterpart each bind CXCR4 with high affinity but only the monomer induces chemotactic cell migration. In contrast, the engineered CXCL12 locked dimer molecule binds to CXCR4 at physiologic concentrations and stimulates an active non-motile state in vitro that disrupts the metastasis of tumor cells in vivo.

CXCR4 responds differently to the monomer and dimer forms of CXCL12. The efficacy of the engineered CXCL12 locked dimer in blocking the homing of metastatic tumor cells or HSCs may be a consequence of agonist-biased CXCR4 signaling or competitive inhibition. Agonist signaling by the CXCL12 locked dimer may induce a non-motile state wherein cells are refractory to other ligands activating CXCR4. In contrast to plerixafor/AMD3100, the engineered CXCL12 locked dimer activates a subset of the signaling pathways stimulated by wild-type CXCL12 and leads to internalization of the receptor from the cell surface. By rendering them insensitive to the attractive CXCL12 signals in the bone marrow, the engineered CXCL12 locked dimer will be significantly more effective at HSC and multiple myeloma tumor cell mobilization than plerixafor/AMD3100.

Chemokines are subject to digestion by extracellular proteases (13). The natural CXCL12 protein is rapidly inactivated in serum by dipeptidyl peptidase-IV, MMP-2 and other enzymes that cleave amino acids at the amino terminus (14, 15). We discovered that the CXCL12 locked dimer has enhanced stability against enzymatic digestion in serum relative to the natural CXCL12 chemokine (10).

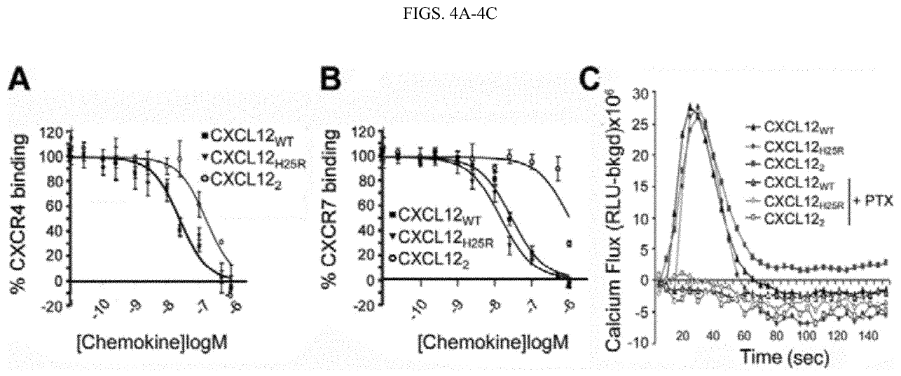

The engineered CXCL12 locked dimer binds CXCR4 at physiologically low nanomolar concentrations and activates the expected and prototypical G protein dependent intracellular calcium flux (FIGS. 4A-4C). We have found that CXCL12 locked dimer receptor binding activates a bioenergetic molecular brake that halts cell migration and renders cells incapable of responding to migration-inducing CXCR4 ligand (9, 11, 16). CXCL12 locked dimer stimulated cells are still able to migrate in response to other, non-CXCR4, chemokine ligands. In side-by-side equimolar comparison experiments CXCL12 locked dimer blocked tumor metastasis at doses where plerixafor/AMD3100 was ineffective (FIGS. 5A-5B) (10).

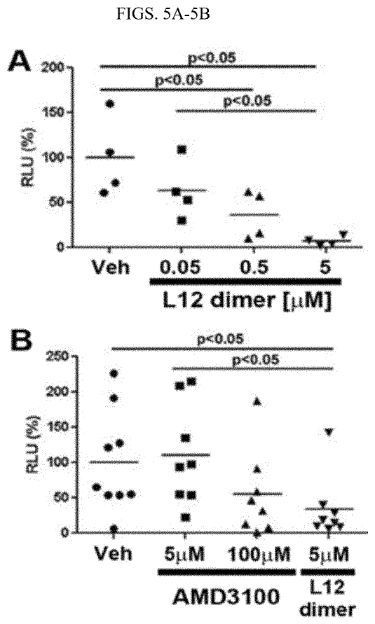

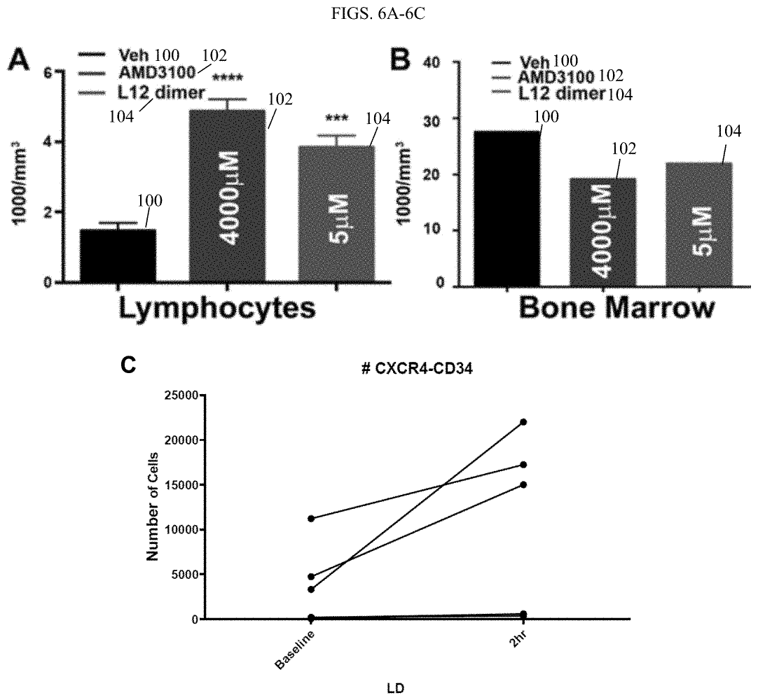

The chemokine CXCL12 is produced by bone marrow stromal cells (17). A single dose infusion of plerixafor/AMD3100 competitively antagonizes CXCR4 to disrupt stromal CXCL12 interactions with bone marrow cells, mobilizing HSC to enter into the peripheral blood (6). We found that 5 μM CXCL12 locked dimer potently stimulated mobilization of hematopoietic cells into peripheral circulation (FIG. 6A) with a concomitant decrease in bone marrow resident lymphocytes (FIG. 6B). Importantly, the 5 μM the CXCL12 locked dimer dose used is the same concentration we have used to block tumor metastasis in vivo and where plerixafor/AMD3100 is ineffective (FIGS. 5A-5B). These data indicate that CXCL12 locked dimer is an immune stimulant capable of mobilizing hematopoietic cells out of the bone marrow.

The CXCL12 locked dimer and mobilizing leukocytes and hematopoietic stem cells—The CXCL12-α2 locked dimer is 1000× more potent on a molar basis (and 3X more potent on a mg/kg basis) than plerixafor for mobilizing lymphocytes (FIGS. 6A-6B) as well as monocytes and neutrophils (not shown). FIG. 6C demonstrates mobilization of CXCR4+ CD34+ hematopoietic stem cells. After blood collection, treated and control mice were sacrificed and the bone marrow of each femur flushed with PBS. Peripheral blood and marrow samples were centrifuged (1800×g for 15 min at room temperature), the plasma removed, and the leukocytes in the buffy coat collected, an aliquot used to enumerate the total cell population using hemocytometry, and the remaining cells transferred to flow cytometry tubes. Peripheral and bone marrow immunocytes were incubated with fluorescence-tagged antibodies to detect the HSC markers CD34, CD11, SCA1, CD90, CD38, Lin and CXCR4, or the T cell and B cell markers CD3 and CD19, respectively.

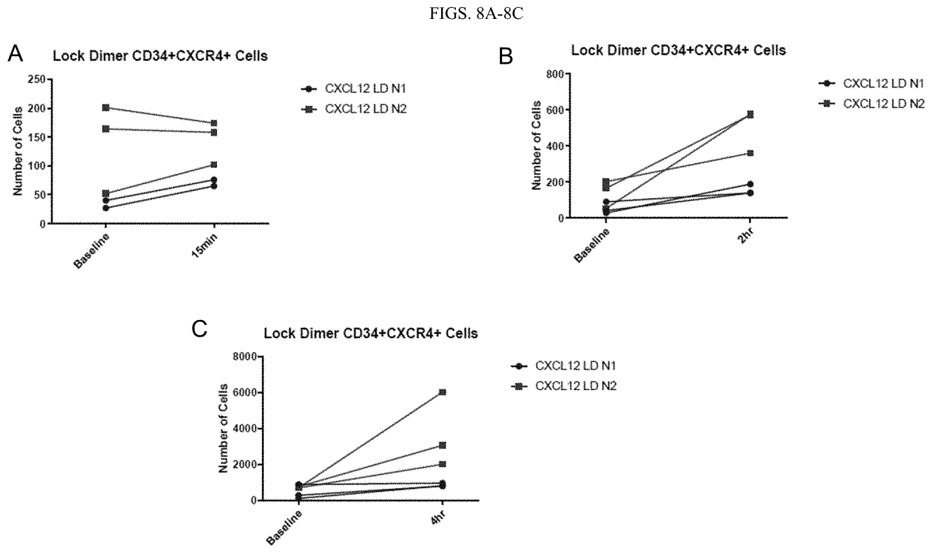

FIGS. 8A-8C also show mobilization of hematopoietic stem cells by the CXCL12 locked dimer. Mice were bled (baseline) prior to subcutaneous injection of the CXCL12 locked dimer (LD) at a final concentration of 5 μM. Mice were re-bled 15-minutes (FIG. 8A), 2-hours (FIG. 8B) or 4-hours (FIG. 8C) later. Erythrocytes were lysed and the leukocytes in the buffy coat collected and immunostained with antibodies to the HSC marker CD34 and the locked dimer receptor CXCR4. Levels of CD34+CXCR4+ stem cells within circulating blood were detected and quantified using multiparametric flow cytometry. Two separate cohorts of mice (shown by circles and squares in FIGS. 8A-8C) were treated (n=3 per group) and show consistent increases in the number of CD34+ stem cells in circulation.

Effect of the CXCL12 locked dimer vs plerixafor (AMD3100) on hematopoietic cell mobilization—As demonstrated in FIGS. 6A-6B, there was no statistical difference between CXCL12 locked dimer 104 and AMD3100 102 in the ability to mobilize lymphocytes into the peripheral blood (A) and out of the bone marrow (B) 1 hour after subcutaneous injection of 5 μM (0.8 mg/kg) CXCL12 locked dimer or 4000 μM (20 mg/kg) plerixafor/AMD3100. Control mice received an injection of the chemokine vehicle (Veh), sterile PBS 100. Values are mean ±SEM, n=3-4 mice per group. Asterisks (****)=P≤0.001.

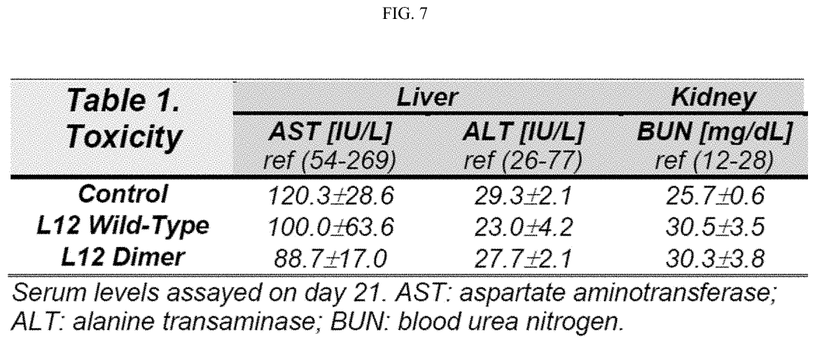

Toxicity—Blood, bone marrow, and tissue levels of CXCL12 were measured using a commercially available sandwich ELISA assay (20-22). The ELISA will detect both endogenous and exogenous CXCL12. Toxicity is monitored using histopathologic assessment of heart, and kidney, and serum assays of hepatic (e.g. AST, ALT, AP) and kidney (e.g. BUN, creatinine) biomarkers, using standard veterinary assays. Data in mice shown in FIG. 7 indicate that twice weekly treatment with 5 μM with either wild-type CXCL12 or CXCL12 locked dimer for 3 weeks resulted in little hepatic or kidney toxicity. Published data (Roy et al., 2014) indicate twice weekly injection of 5 μM chemokine protein is well tolerated, with no change in body weight or appearance after 120 days in tumor xenografted mice.

### REFERENCES

(1) Chen B J, Cui X, Sempowski G D, Domen J, Chao N J. Hematopoietic stem cell dose correlates with the speed of immune reconstitution after stem cell transplantation. Blood 2004;103:4344-52.

(2) Halter J, Kodera Y, Ispizua A U, Greinix H T, Schmitz N, Favre G, et al. Severe events in donors after allogeneic hematopoietic stem cell donation. Haematologica 2009;94:94-101.

(3) Deol A, Abrams J, Masood A, Al-Kadhimi Z, Abidi M H, Ayash L, et al. Long-term follow up of patients proceeding to transplant using plerixafor mobilized stem cells and incidence of secondary myelodysplastic syndrome/AMI. Bone Marrow Transplant 2013;48:1112-6.

(4) Tichopad A, Vitova V, Koristek Z, Lysak D. Cost-effectiveness of hematopoietic stem cell mobilization strategies including plerixafor in multiple myeloma and lymphoma patients. J Clin Apher 2013;28:395-403.

(5) Tzeng Y S, Li H, Kang Y L, Chen W C, Cheng W C, Lai D M. Loss of Cxcl12/Sdf-1 in adult mice decreases the quiescent state of hematopoietic stem/progenitor cells and alters the pattern of hematopoietic regeneration after myelosuppression. Blood 2011;117:429-39.

(6) Broxmeyer H E, Orschell C M, Clapp D W, Hangoc G, Cooper S, Plett P A, et al. Rapid mobilization of murine and human hematopoietic stem and progenitor cells with AMD3100, a CXCR4 antagonist. J Exp Med 2005;201:1307-18.

(7) Salman H, Lazarus H M. Plerixafor for autologous CD34 cell mobilization. Core Evid 2011;6:23-9.

(8) Veldkamp C T, Seibert C, Peterson F C, De la Cruz N B, Haugner J C, III, Basnet H, et al. Structural basis of CXCR4 sulfotyrosine recognition by the chemokine SDF-1/CXCL12. Sci Signal 2008;1:ra4.

(9) Drury L J, Ziarek J J, Gravel S, Veldkamp C T, Takekoshi T, Hwang S T, et al. Monomeric and dimeric CXCL12 inhibit metastasis through distinct CXCR4 interactions and signaling pathways. Proc Natl Acad Sci U S A 2011;108:17655-60.

(10) Takekoshi T, Ziarek J J, Volkman B F, Hwang S T. A locked, dimeric CXCL12 variant effectively inhibits pulmonary metastasis of CXCR4-expressing melanoma cells due to enhanced serum stability. Mol Cancer Ther 2012;11:2516-25.

(11) Roy, McAllister D, Gorse E, Dixon K, Piper C T, Zimmerman N P, et al. Pancreatic Cancer Cell Migration and Metastasis Is Regulated by Chemokine-Biased Agonism and Bioenergetic Signaling. Cancer Res 2015;75:3529-42.

(12) Andresen B T. A pharmacological primer of biased agonism. Endocr Metab Immune Disord Drug Targets 2011;11:92-8.

(13) Overall C M, McQuibban G A, Clark-Lewis I. Discovery of chemokine substrates for matrix metalloproteinases by exosite scanning: a new tool for degradomics. Biol Chem 2002;383:1059-66.

(14) Christopherson K W, Hangoc G, Broxmeyer H E. Cell surface peptidase CD26/dipeptidylpeptidase IV regulates CXCL12/stromal cell-derived factor-1 alpha-mediated chemotaxis of human cord blood CD34+ progenitor cells. J Immunol 2002;169:7000-8.

(15) McQuibban G A, Butler G S, Gong J H, Bendall L, Power C, Clark-Lewis I, et al. Matrix metalloproteinase activity inactivates the CXC chemokine stromal cell-derived factor-1. J Biol Chem 2001;276:43503-8.

(16) Veldkamp C T, Ziarek J J, Su J, Basnet H, Lennertz R, Weiner J J, et al. Monomeric structure of the cardioprotective chemokine SDF-1/CXCL12. Protein Sci 2009;18:1359-69.

(17) Nagasawa T, Kikutani H, Kishimoto T. Molecular cloning and structure of a pre-B-cell growth-stimulating factor. Proc Natl Acad Sci U S A 1994;91:2305-9.

(18) Segers V F, Revin V, Wu W, Qiu H, Yan Z, Lee R T, et al. Protease-resistant stromal cell-derived factor-1 for the treatment of experimental peripheral artery disease. Circulation 2011;123:1306-15.

(19) Ziarek J J, Veldkamp C T, Zhang F, Murray N J, Kartz G A, Liang X, et al. Heparin oligosaccharides inhibit chemokine (CXC motif) ligand 12 (CXCL12) cardioprotection by binding orthogonal to the dimerization interface, promoting oligomerization, and competing with the chemokine (CXC motif) receptor 4 (CXCR4) N terminus. J Biol Chem 2013;288:737-46.

(20) Wendt M K, Johanesen P A, Kang-Decker N, Binion D G, Shah V, Dwinell M B. Silencing of epithelial CXCL12 expression by DNA hypermethylation promotes colonic carcinoma metastasis. Oncogene 2006;25:4986-97.

(21) Wendt M K, Cooper A N, Dwinell M B. Epigenetic silencing of CXCL12 increases the metastatic potential of mammary carcinoma cells. Oncogene 2008;27:1461-71.

(22) Roy I, Zimmerman N P, Mackinnon A C, Tsai S, Evans D B, Dwinell M B. CXCL12 chemokine expression suppresses human pancreatic cancer growth and metastasis. PLoS ONE 2014;9:e90400.

(23) Thomas M A, Buelow B J, Nevins A M, Jones S E, Peterson F C, Gundry R L, et al. Structure-function analysis of CCL28 in the development of post-viral asthma. J Biol Chem 2015;290:4528-36.

We claim:

1. A method of mobilizing hematopoietic stem cells into the bloodstream of a subject, the method comprising:
administering to the subject a therapeutically effective amount of a composition comprising a CXCL12-α2 locked dimer.

2. The method of claim 1, wherein the CXCL12-α2 locked dimer comprises two monomers locked together by one or more disulfide linkages.

3. The method of claim 1, wherein at least one of the monomers has the amino acid sequence of SEQ ID NO:1.

4. The method of claim 3, wherein the CXCL12- α2 locked dimer is locked together by disulfide linkages at residues 36 and 65 of at least one of the monomers having the sequence of SEQ ID NO:1.

5. The method of claim 1, wherein the monomers are identical.

6. The method of claim 1, wherein the monomers are not identical.

7. The method of claim 1, additionally comprising the step of harvesting the hematopoietic cells from the subject by apheresis.

8. The method of claim 1, wherein the subject is human.

9. A method of mobilizing cancer cells into the bloodstream of a subject, the method comprising:
administering to the subject a therapeutically effective amount of a composition comprising a CXCL12-α2 locked dimer.

10. The method of claim 9, wherein at least one of the monomers has the amino acid sequence of SEQ ID NO:1.

11. The method of claim 10, wherein the CXCL12-α2 locked dimer is locked together by disulfide linkages at residues 36 and 65 of at least one of the monomers having the sequence of SEQ ID NO:1.

12. The method of claim 9, wherein the monomers are identical.

13. The method of claim 9, wherein the monomers are not identical.

14. The method of claim 9, wherein the subject is human.

15. The method of claim 9, wherein the cancer cells express CXCR4.

16. A method of treating blood cancer in a subject, the method comprising:
administering to the subject a therapeutically effective amount of a composition comprising a CXCL12-α2 locked dimer, and
administering to the subject a therapeutically effective amount of a chemotherapeutic agent.

17. The method of claim 16, wherein at least one of the monomers has the amino acid sequence of SEQ ID NO:1.

18. The method of claim 16, wherein the chemotherapeutic agent is selected from the group consisting of fludarabine, idarubicin, cytarabine, etoposide, cladribine, mitoxantrone, and venetoclax.

19. The method of claim 16, wherein the cancer is selected from the group consisting of leukemia, lymphoma, and myeloma.

20. The method of claim 16, wherein the cancer expresses CXCR4.
